(19) United States
(12) Patent Application Publication
(10) Pub. No.: US 2008/0199915 A1
(43) Pub. Date: Aug. 21, 2008

(54) Methods and Kits For Mass Production Of Dsrna

(75) Inventors: Eugene Makeyev, Cambridge, MA (US); Dennis Bamford, Helsinki (FI)

Correspondence Address: FOLEY AND LARDNER LLP;SUITE 500, 3000 K STREET NW, WASHINGTON, DC, 20007, US

(73) Assignee: RNA-Line Oy

(21) Appl. No.: 10/559,576

(22) PCT Filed: May 2, 2003
(86) PCT No.: PCT/FI2004/000347

§ 371 Date: Feb. 8, 2008

(30) Foreign Application Priority Data
Jun. 6, 2003 (FI) 20030854

(51) Int. Cl. C12P 19/34; C12M 1/00; C12N 15/87

(52) U.S. Cl. 435/91.2; 435/289.1; 435/455

(57) ABSTRACT

The invention relates to methods and kits for propagating target nucleic acid in the form of double stranded RNA. This invention relates in particular to a method for mass production of dsRNA. The method comprises that a target nucleic acid is provided in a form replicable by an RNA-dependent RNA polymerase in a living cell, said replicable form of the target nucleic acid is contacted with said polymerase under conditions sufficient for template-directed RNA synthesis, wherein one of the reaction products is necessarily double-stranded (ds) RNA and said dsRNA products are recovered in a sufficiently pure form. The dsRNA products can be used in various applications, for example in gene silencing.

### FIELD OF THE INVENTION

The present invention relates to the field of mass production of dsRNA. This invention relates also to a living cell system and to a kit for mass production of dsRNA. More specifically this invention relates to the use of RNA viruses and other RNA replicons for providing target nucleic acid sequences in the form of dsRNA.

### BACKGROUND OF THE INVENTION

The methods to produce large amounts of DNA are well developed and widely used. The recent developments in molecular and cellular biology has revealed that RNA molecules in general and dsRNA molecules in particular play a much more central role in a number of cellular processes than previously was known. One of such processes is posttranscriptional gene silencing. This progress obviously leads to the need of large-scale production methods for sequence specific dsRNA production. Currently the favorite method in use is the plasmid derived ssRNA synthesis followed by annealing of two complementary RNA molecules to gain dsRNA. Although such technology is successful it is difficult to produce long RNA molecules and the method is not practical and not cost effective for producing industrial quantities of dsRNA. When therapeutic and diagnostic use of dsRNA is needed, a reliable, low cost, high quantity (grams) method with a capacity to produce several kbp long dsRNA molecules is requested.

In this invention it has been found that dsRNA can be produced by using a RNA-dependent RNA polymerase, in particular from an RNA virus or replicon, in a living cell with unexpectedly high yield. In the literature (Onodera et al. 1992) it has been shown that a marker antibiotic resistance gene can be inserted to the dsRNA bacteriophage in a dsRNA form and that such intracellular viral elements confer the cells resistant to the encoded antibiotic. However, it has not been suggested to use dsRNA bacteriophages or other RNA replicons for amplifying a nucleic acid target of interest and no one has suggested the use of dsRNA bacteriophages or other RNA replicons for mass production of a target dsRNA. As discussed above, the existing in vitro methods for generating dsRNAs can provide only limited amounts of the product. It is therefore advantageous to develop a method wherein dsRNA can be produced from a renewable source, such as living cell, and purified using a straightforward procedure. Toward this end, the present invention offers a simple and convenient strategy wherein RNA replicons (such as RNA viruses, RNA virus-like particles, RNA plasmids, or derivatives thereof) are used to propagate target nucleic acid sequences in the form of dsRNA.

### SUMMARY

In a first aspect, the present invention provides a novel method for mass production of dsRNA. It is based on the use of an RNA-dependent RNA polymerase, from viruses or other types of replicons with dsRNA genome, with which it is easy to produce dsRNA of sufficient purity and in sufficient amounts. More specifically, the method is mainly characterized by what is stated in the characterizing part of claim 1.

In a second aspect, the present invention provides a living cell system for mass production of dsRNA. The living cell system is mainly characterized by what is stated in the characterizing part of claim 16.

In a third aspect, the present invention provides a kit for mass production of dsRNA. The kit is mainly characterized by what is stated in the characterizing part of claim 22.

In a fourth aspect, the present invention provides a method for inducing sequence-specific gene silencing in eukaryotic organisms based on RNA viruses or other RNA replicons. In the method, RNA replicons are used as vehicles for propagating target nucleic sequences in a dsRNA form; the dsRNA is purified and used to trigger silencing effects. More specifically, the method is mainly characterized by what is stated in the characterizing part of claim 23.

According to a preferred embodiment the present invention provides a method where viral replication complexes in carrier state microbial cells produce practically unlimited amounts (fermentor scale) of dsRNA. Desired nucleic acid sequences can be transformed in a vector to the carrier state cells where the transient transcription produces desired ssRNA molecules. These are directed for packaging into the intracellular viral replication complexes where the complementary strand is synthesized. After propagation of such cells dsRNA of interest can be isolated and purified.

A number of biological entities having RNA genomes will be appropriate systems for the use within this methodology. For example, at least some ssRNA viruses are known to replicate their genomes via dsRNA intermediates (Buck, 1996). However, for the ease of obtaining dsRNA of sufficient purity and in sufficient amounts it is advantageous to use viruses or other types of replicons with dsRNA genomes.

One can make use of essentially any RNA-based organism or system, including RNA virus-like particles, RNA plasmids, viroids, or other RNA-based autonomous genetic elements. According to a preferred embodiment of the invention the RNA based system is an RNA bacteriophage which belongs to Cystoviridae family, preferably the bacteriophage is selected from the group of φ6, φ7, φ8, φ9, φ10, φ11, φ12, φ13 and φ14, most preferably from bacteriophage φ6. The replicable form of the nucleic acid target is contacted with the polymerase in a prokaryotic cell, preferably in a gram-negative bacterial cell, more preferably in a bacterial cell selected from the group comprising Pseudomonas sp., Escherichia sp. and Salmonella sp., most preferably in a cell of Pseudomonas syringae. A currently preferred embodiment rely on a genetically altered bacteriophage φ6, a dsRNA virus from the Cystoviridae family that infects the bacterium Pseudomonas, in particular P. syringae (Mindich, 1988; Mindich, 1999a).

The present invention provides also a novel method for constructing recombinant dsRNA bacteriophages. The method takes advantage of suicide vectors wherein nucleic acid fragments of interest are operably linked with the sequences sufficient for detectable replication by the viral replication apparatus. The new method is faster and easier than previously described methods for constructing recombinant dsRNA bacteriophages, which involve in vitro packaging of procapsids particles (Poranen et al., 2001) or propagating genetically modified bacteriophages in host cells stably transformed with the plasmid expressing target genes (Mindich, 1999b) and references therein).

In the currently preferred embodiment said suicide vector is a DNA plasmid that is delivered into a cell containing functional viral replication apparatus. The plasmid can not be stably propagated within said cell (definition of a suicide vector), but can be transiently transcribed by a DNA-dependent RNA polymerase to yield RNAs replicable by the viral polymerase.

Because RNAs replicable by dsRNA virus polymerase in vivo are converted into dsRNA (genomic) form, the use of the suicide vector strategy is highly advantageous for various applications of this invention.

The present invention is of great advantage since it provides tools for the continuously growing research on the RNA field. From the possible application areas the use of dsRNA in gene silencing is at the moment most promising.

Further features, aspects and advantages of the present invention will be better understood from the description of specific embodiments and examples. It should be understood, however, that the description and the examples are given by the way of illustration only, not by the way of limitation. Various changes and modifications within the spirit and the scope of the invention will become apparent to those skilled in the art from the following text. Furthermore, citation of a reference throughout the entire patent text shall not be interpreted as an admission that such is prior art to the present invention.

### BRIEF DESCRIPTION OF THE FIGURES

The foregoing text, as well as the following description and appended claims, will be better understood when read in conjunction with the appended figures, in which:

FIG. 1 shows schematically how recombinant RNA replicons are generated using suicide plasmid strategy. The example depicts constructing carrier-state Pseudomonas syringae cells that contain recombinant 6 virus expressing beta-lactamase gene (φ6-bla).

FIG. 2 depicts:

(A) Agarose gel electrophoresis of total RNA from the following strains: K, Km-resistant HB10Y(φ6-npt); A0, Amp-resistant HB10Y(φ6-bla); HB, non-infected HB10Y. Lane φ6, dsRNA segments L, M and S extracted from the wild-type φ6 (positions indicated on the left along with the positions of P. syringae 23S and 16S rRNAs). Mk, dsDNA markers. Marker lengths in kbp are shown on the right. White arrowhead shows the new segment, M-bla, which appears in Amp-resistant cells.

(B) RT-PCR analysis with npt- and bla-specific primers was performed using RNA from: K, HB10Y(φ6-npt) and A0, HB10Y(φ6-bla). The reverse transcription (RT) step was omitted in reactions 2 and 5. Different PCR primers were used as specified under the panel. Positions of the npt and bla-specific PCR fragments are marked on the right. dsDNA marker (Mk) lengths are shown on the left.

FIG. 3 shows that φ6-bla carrier cells rapidly adapt to cefotaxime.

(A) 0.2 to 1×10⁷ HB10Y(φ6-bla) carrier state cells were plated onto LB agar containing either 150 μg/ml ampicillin (Amp150) or 50 μg/ml cefotaxime (Ctx50). Ctx resistant colonies appeared after 3 days of incubation at 28° C. No colonies were detected at this time on the sector inoculated with 1×10⁷ HB10Y(pLM254) cells, which contain a plasmid encoding the bla gene.

(B) Schematic diagram of the Ctx adaptation experiment. Cells were cultivated on LB agar containing increasing Ctx concentrations (μg/ml), as shown below petri dishes. 20-40 of the largest colonies were pooled after each passage and used for subsequent rounds of selection.

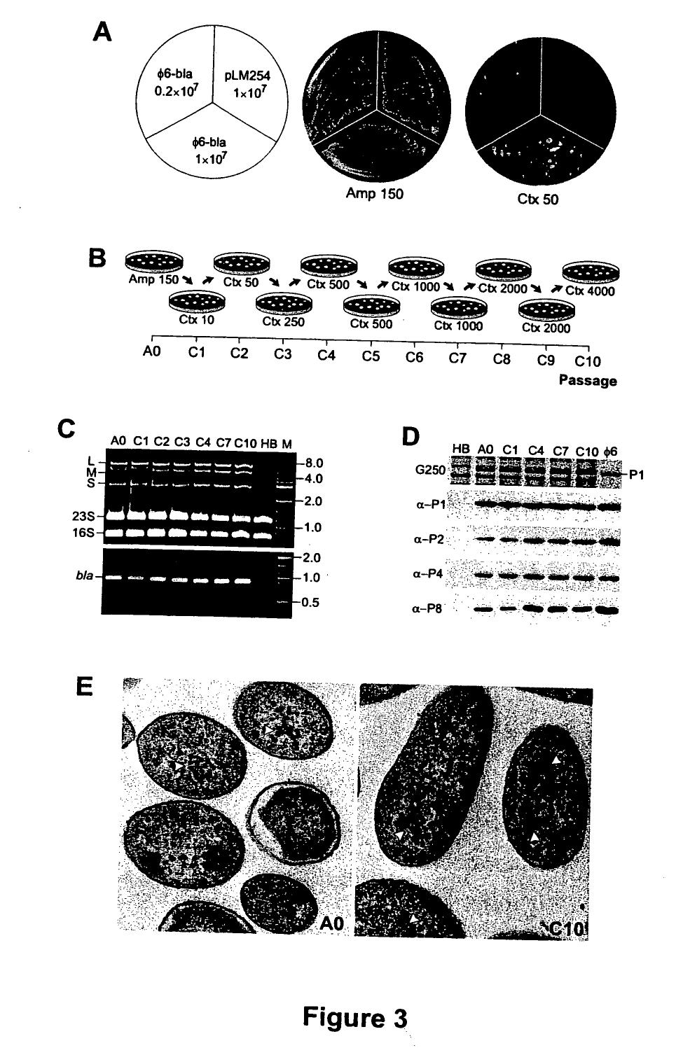

(C) Upper panel, agarose gel analysis of RNA extracted from carrier state cells at passages A0, C1, C2, C3, C4, C7 and C10. HB, RNA from uninfected HB10Y cells. Lower panel, RT-PCR products generated using bla-specific primers. Other designations are as defined in the description of FIG. 2.

(D) SDS-PAGE analysis (Olkkonen and Bamford, 1989) of carrier state cells from different passages (A0, C1, C4, C7 and C10) or purified φ6 virus (φ6). HB, uninfected HB10Y cells. Panel G250, a Coomassie G250 stained gel fragment showing the band of protein P1. α-P1, α-P2, α-P4, and α-P8, immunoblots produced using antibodies specific to corresponding φ6 nucleocapsid (NC) proteins and ECL detection as recommended by Pierce Biotechnology.

(E) Transmission electron micrograph of osmium tetroxide and uranyl acetate stained cell thin sections from A0 and C10 passages taken as described (Bamford and Mindich, 1980). Black arrowhead, enveloped virions; white arrowhead, NC and PC particles.

### DETAILED DESCRIPTION OF THE INVENTION

### Definitions

Unless explicitly stated otherwise, specific terms used throughout this invention have the following meanings:

The term “bacteriophage” refers to a virus infecting eubacteria or another prokaryotic organism, such as e.g. archaea.

The term “biological activity”, as used herein, refers broadly to various functions and properties of a protein or nucleic acid. Examples of biological activities include but are not limited to catalytic, binding, and regulatory functions.

As used herein, the term “biological entity”, refers to all systems containing nucleic acids capable of multiplication through a template-directed mechanism.

As used herein, the term “carrier-state cells” refers to a cell line or plurality of cells infected by a virus, which can support multiple rounds of the virus genome replication, remaining in a living state for a period of time substantially longer than a typical duration of the virus life cycle.

The term “nucleic acid sequence”, or sometimes “nucleotide sequence”, refers to an order of nucleotides in an oligonucleotide or polynucleotide chain.

The term “polymerase”, or sometimes “nucleic acid polymerase”, refers to a protein or a protein complex that can catalyze the polymerization of ribo- or deoxyribo-nucleoside triphosphates into a polynucleotide chain.

The term “protein sequence”, or sometimes “amino acid sequence”, refers to an order of amino acid residues in a peptide or protein chain.

As used herein, the term “ribovirus” refers to an RNA virus whose life cycle proceeds entirely on the level of RNA and does not normally include a DNA phase. Riboviruses include viruses with positive- and negative-sense single-stranded (ss) RNA genomes as well as double-stranded (ds) RNA viruses. A preferred embodiment of this invention deals with dsRNA viruses from the Cystoviridae family, also referred to as “cystoviruses”. Also see “RNA virus”. The dsRNA virus is preferably a bacteriophage selected from the group comprising φ6, φ7, φ8, φ9, φ10, φ11, φ12, φ13 and φ14, most preferably it is bacteriophage φ6.

As used herein, the term “reverse-transcribing virus” refers broadly to a virus whose life cycle necessarily includes both RNA and DNA phases. The name of the group derives from the process of “reverse transcription” used by these viruses wherein RNA molecules are used as templates to produce DNA copies. Two types of reverse-transcribing viruses are known, “retroviruses” and “pararetroviruses”. Retroviruses encapsidate their genomes in the form of RNA but use DNA intermediates when multiplying in infected cells. Pararetroviruses encapsidate DNA genomes but use RNA intermadiates when multiplying in infected cells.

The term “ribozyme” refers to an RNA molecule with detectable catalytic activity. Various natural and artificial ribozymes possessing diverse catalytic activities have been described in the previous art (Bittker et al., 2002b; Doudna and Cech, 2002; Jaschke, 2001).

The term “RNA virus” refers to viruses having RNA genomes.

As used herein, the term “RNA-based autonomous genetic element” refers generically to biological entities containing RNA genome but distinct from RNA virus. RNA-based autonomous genetic elements include but are not limited to RNA virus-like particles, viroids, and RNA plasmids. Another term sometimes used in the literature to refer to RNA-based autonomous genetic elements is “RNA subviral agent”. Also see definition of “biological entity”.

The term “RNA-based organism”, as used herein, refers generically to RNA viruses and RNA-based autonomous genetic elements defined above. Because all RNA organisms are capable of replicating their genomes under appropriate conditions, the term “RNA replicon” is used herein in reference to RNA organisms and derivatives thereof to emphasize this capability.

The term “RNA-dependent polymerase” refers to a nucleic acid polymerase capable of copying RNA templates. Two types of RNA-dependent polymerases are known, producing RNA or DNA copies of RNA templates. These are referred to as “RNA-dependent RNA polymerases” (“RdRP”) and “RNA-dependent DNA polymerases” (“RdDP”, better known as reverse transcriptases), respectively. Also see “polymerase”.

As used herein, the terms “target” or “target molecule” refer to a nucleic acid that is subjected to the methods of this invention. Plurality of target molecules comprising one or many distinct variants is sometimes referred to as “target population”. The length of a target nucleic acid can be from about 20 bases, preferably from about 50 bases to 15 kilobases, more preferably it is from 300 bases to 3 kilobases.

“Heterologous target sequence” refers here to a target sequence from any possible origin except from the RNA-based biological entity (e.g. RNA virus), which is used in the replication of the target sequence. “Homologous target sequence” refers here to a target sequence from the RNA-based biological entity (e.g. RNA virus), which is used in the replication of the target sequence.

The target nucleic acid sequence may be homologous or heterologous, in particular it may be heterologous, to the RNA virus or replicon.

“A living cell” refers here to a cell supporting the replication of an RNA-based biological entity, such as RNA virus or other RNA replicon. The living cells may belong to prokaryotes. They may be bacteria, preferably gram-negative bacteria, more preferably bacteria selected from the group comprising Pseudomonas sp., Escherichia sp. and Salmonella sp., most preferably Pseudomonas syringae. The living cell may also be a eukaryotic cell, such as mammalian, insect, plant or yeast cell.

“Detectable replication” refers here to the replication of the nucleic acid target detectable by any standardly available molecular biology method.

As used herein, the term “suicide vector” or a more specific term “suicide plasmid” refer to, respectively, vector/plasmid that can not be stably maintained within given cell line but can direct transient gene expression.

Other terms are explained in the text or used according to the common practices of the art.

### Viral RNA Vectors

In the selected formats, target is integrated within RNA replicons, thus allowing replication of the target by an appropriate RNA-dependent polymerase. It may be advantageous for many applications to choose RNA viruses as RNA replicons. In this case, integrated target is replicated as a part of viral genome by the virus-encoded polymerase, preferably RNA-dependent polymerase. In previous experiments RNA viruses have been used as vectors for heterologous sequence inserts. For example, alphaviruses, retroviruses and some (−)RNA viruses are used as vectors for gene therapy and gene expression application (Palese, 1998; Robbins et al., 1998). Similarly, several RNA viruses infecting plants may also be used as vectors (Lindbo et al., 2001).

Although some embodiments of the method can rely on single-stranded RNA viruses, it may be advantageous for many applications to select viruses that have double-stranded RNA genome. dsRNA resist nuclease degradation better than ssRNA, which makes it easier to purify sufficient amount of intact dsRNA than that of ssRNA. Examples of dsRNA viruses include members of the Cystoviridae, Reoviridae, Totiviridae, Partitiviridae, Birnaviridae and Hypoviridae families. Because of the economical and convenience reasons it may be advantageous to use viruses from the Cysto-, Toti- and Partitiviridae families, which infect prokaryotes and lower eukaryotic organisms such as bacteria, yeast and other fungi. Bacteriophage φ6 and its relatives (φ7 through φ14) infecting gram-negative bacteria and Saccharomyces cerevisiae viruses L-A and L-BC, that have been also known under the name of “virus-like particles”, are amongst the most obvious choices.

In the currently preferred embodiment, target gene is integrated within the genomic RNA of a dsRNA bacteriophage from the Cystoviridae family (a cystovirus). An important advantage of an RNA bacteriophage over animal or plant RNA viruses is the low cost and relative ease of propagation. Furthermore, bacteriophages generally have shorter life cycles, which helps to reduce the time needed for the production.

As a specific example of the dsRNA bacteriophage format, target gene can be integrated into the M segment of the cystovirus φ6 and replicated by the φ6-encoded RNA-dependent RNA polymerase. In further embodiments, other members of the Cystoviriae family, from φ7 through φ14 (Mindich et al., 1999), can be used as vectors for target sequences and also as polymerase source. Furthermore, any of the three genomic segments L, M and S, typical for the Cystoviridae, can be used for integrating the target sequence.

Furthermore, it is known that at least some cystoviruses can tolerate substantial genome rearrangements, which can be manifested in the form of shortened or extended genomic segments, or a change in the segment number. For example, variants of φ6 containing 1, 2 or 4 genomic segments have been described (Onodera et al., 1995; Onodera et al., 1998). These modified cystoviruses are also within the scope of this invention, as they can be more advantageous RNA vectors than the wild-type cystoviruses.

It has been shown that the synthesis of cystoviral RNA is catalyzed by so-called polymerase complex that includes proteins P1, P2 (catalytic subunit), P4, and P7 (Mindich, 1999a; Mindich, 1999b). The polymerase complex also serves as a container for genomic RNA. All polymerase complex proteins are encoded on the segment L. Earlier studies have also demonstrated that bacterial cells expressing cDNA of the L segment accumulate functional polymerase complex particles (Mindich, 1999b). Therefore, some embodiments may involve the use of cystovirus derivatives whose L segment encodes for the polymerase complex, whereas additional segment(s) are used for incorporating nucleic acid targets. In alternative embodiments, proteins of the polymerase complex can be produced from cDNA, which can be introduced into bacterial cell for example in the form of a DNA plasmid. In this case, the entire genetic capacity of the polymerase complex (˜15 kb) can be used for dsRNA production with a specific sequence.

It is a currently preferred feature that the RNA virus vector used is propagated in the form of carrier state cells. This type of viral infection does not destroy most of the infected cells, thus effectively extending time of the target gene expression. Clearly, all formats where virus is not lethal for the infected cell will be particularly useful for the dsRNA production. In the currently preferred embodiment, recombinant bacteriophage φ6 is propagated within carrier-state bacteria Pseudomonas syringae. Because at least some of the related cystoviruses have been shown to infect Escherichia coli and Salmonella typhimurium (Hoogstraten et al., 2000; Mindich et al., 1999; Qiao et al., 2000), additional embodiments of this invention will be based on the use of carrier-state gram-negative bacteria containing a recombinant cystovirus selected from the group of φ6, φ7, φ8, φ9, φ10, φ11, φ12, φ13, and φ14.

In further specific embodiments, non-lethal infection can be achieved by using special cell lines, weakened (attenuated) virus strains, or both. As an example of the first strategy, mutants of P. syringae cells are known that form carrier state cells after being infected with the wild-type φ6 virus. Attenuated viruses can be selected as naturally occurring mutants or engineered artificially. In some cases it will be sufficient to substitute a part of viral genes with the target sequence to obtain an attenuated virus. Interestingly, non-lethal infection is typical for the normal life cycles of several viruses. The examples include above-mentioned yeast totiviruses L-A and L-BC.

### Non-Viral RNA Vectors

Although the use of virus-based vectors is advantageous for many applications, some embodiments may use non-viral vectors. One example of this strategy is to use specific elements that are replicated in nature by viral RNA-dependent RNA polymerases, such as diverse defective interfering (DI) elements and satellite RNAs. Specific examples include small RNAs multiplied by the RdRP of the coliphage Qβ and toxin-encoding satellites of the yeast L-A virus (M1, M2, and others) (Brown and Gold, 1995; Wickner, 1996).

Another example of non-viral vectors would be the use of autonomous genetic elements found for example in fungi and plants. S. cerevisiae strains often contain single-stranded replicons called 20S RNA and 23S RNA. Of these, 20S RNA is an apparently naked RNA replicon (with a dsRNA form called W) encoding an RNA polymerase. 23S RNA also encodes an RNA polymerase and has a dsRNA form called T (Wickner, 1996). Furthermore, some plants, such as rice, are infected by extensive dsRNA elements, referred to as “RNA plasmids” or “endomaviruses” by different authors (Gibbs et al., 2000). These elements encode their own RdRP and seem to lack coat proteins. Many RNA replicons of the non-virus origin normally do not destroy the infected cell, which can be an advantageous feature as discussed above.

### Polymerase Sources

In the aforementioned embodiments, target nucleic acid, integrated into viral or non-viral RNA vector, is replicated by an RNA-dependent polymerase. It will be obvious for those skilled in the art that said polymerase can be provided in any number of ways. In some embodiments, the polymerase will be encoded by the RNA replicon containing the nucleic acid, whereas in other embodiments the polymerase will be encoded by another RNA replicon co-infecting the host cell.

In yet further embodiments, the polymerase can be encoded by DNA, which can be of chromosomal, plasmid, viral, transposon or other origin. An example of this format was discussed above for cystovirus-based vectors. In another specific embodiment, target sequence can be incorporated into viroid RNA and the replication of the genetically altered viroid RNA is probably carried out by cellular RNA polymerase II, operating in this case in the RNA-dependent mode (Lai, 1995). In other embodiments, viral polymerase genes can be introduced in a DNA form into the host cell and expressed using cellular transcription and translation apparatus.

### Delivery Methods

Another important aspect of the methods for mass production of dsRNA is the procedure used for bringing nucleic acid targets in contact with the polymerase.

In a specific embodiment of this invention, this task can be accomplished by contacting a replicable form of the nucleic acid target with said polymerase within living cell. For this purpose, both target and the polymerase have to be delivered into the host cell.

Different delivery methods can be used in different embodiments, ranging from delivery through virus infection, transformation (in bacteria), transfection (in eukaryotic cell lines), electroporation, lipofection, ballistic methods, agroinfiltration, microinjection etc. Description of these and other delivery methods can be found elsewhere.

In the currently preferred embodiment, illustrated in the Example 1, bacteriophage φ6 RdRP is delivered into the host P. syringae cell using virus infection. The heterologous sequence is delivered either through virus infection (as in the φ6-npt case) or in the form of a suicide DNA plasmid using electroporation (as in the φ6-bla case).

In many embodiments, it may be advantageous to deliver RNA replicons containing marker genes. Such marker genes can be very useful to distinguish between cells that contain RNA replicon from the rest of the cells. Indeed, currently available delivery methods may not be 100% efficient, in that only a fraction of the treated cells usually receive the RNA replicon encoding the nucleic acid target. Examples of marker genes may include antibiotic or toxin resistance genes, genes encoding enzymes of amino acid or nucleotide metabolism, or genes encoding fluorescent proteins.

Method for Mass Production of dsRNA

This invention provides a method, wherein RNA replicons are utilized as vehicles for mass production of heterologous or homologous sequences in the dsRNA form in vivo.

This method comprises the steps of:
a) providing nucleic acid target in a form replicable by an RNA-dependent RNA polymerase in a living cell;
b) contacting said replicable form of the nucleic acid target with said polymerase under conditions sufficient for template-directed RNA synthesis, one of the reaction products being necessarily double-stranded (ds) RNA;
c) recovering said dsRNA products in a sufficiently pure form; and optionally modifying said products for optimal performance.

Two major requirements affect the choice of preferred embodiments.

(1) It is advantageous to produce large amount of sufficiently pure dsRNA molecules without substantial expenses.
(2) It is also advantageous to perform all the method steps within shortest time possible.

Accordingly, the currently preferred embodiments of the method utilize recombinant dsRNA viruses infecting prokaryotic and lower eukaryotic organisms, such as Cystoviridae, Totiviridae and Partitiviride. The hosts of these viruses, usually bacteria and fungi, can be propagated easily and inexpensively, thus enabling a mass production of dsRNA from the corresponding recombinant virus. In the most preferred embodiment, dsRNA viruses from the Cystoviridae family are used as vectors for propagating heterologous sequences in the dsRNA form. Other embodiments can certainly make use of other viruses, both of dsRNA and ssRNA nature. The use of ssRNA viruses is theoretically justified since many of these viruses form dsRNA replication intermediates.

It is furthermore preferred that the target sequence to be converted and further propagated in the form of dsRNA is delivered into the host cell in the form of a DNA vector under the control of an appropriate DNA-dependent RNA polymerase promoter. The transcription product derived from said DNA vector must comprise the nucleic acid target and the sequences sufficient for RNA replication. The host cell must contain RNA-dependent RNA polymerase that can replicate the target RNA molecule.

In the currently preferred embodiment target sequence is delivered into P. syringae carrier state cells carrying φ6 virus, in the form of a suicide DNA plasmid that can not be stably propagated in Pseudomonas but can be transiently transcribed by the cellular RNA polymerase. The target is physically linked with a marker gene such as ampicillin of cefotaxime resistance gene, and therefore need not encode for any detectable activity. The translation of the target sequence into protein is also optional. The cells that acquired the target molecule in the form replicable by φ6 polymerase complex will express the marker gene and will be distinguishable from the rest of the cells (e.g. will be ampicillin/cefitaxime resistant).

Further specific embodiments of this invention are based on the use of other recombinant cystoviruses (φ7 through φ14) propagated within carrier-state Pseudomonas sp. or other gram-negative bacteria, such as Escherichia coli or Salmonella typhimurium.

Because it is advantageous that the target sequence is not changed substantially when propagated in the form of dsRNA, in the preferred embodiments, the time of RNA replicon propagation is limited to minimum. In the most preferred embodiment, RNA replicon is propagated within appropriate cell line during 12-96 hours, preferably 24-48 hours.

In the currently selected embodiment, dsRNA is recovered from the carrier state cells using a specific phenol/chloroform extraction and precipitation procedure described in the Example 3. However, other well known methods as well as commercial kits for dsRNA recovery are available. Thus obtained dsRNA preparation may contain ribosomal RNA, tRNA, traces of the bacterial chromosome and proteins. It may therefore be advisable for dsRNA quality sensitive applications to amend this purification procedure with steps removing dsDNA, ssRNA and protein impurities. These steps may include but are not limited to purification using anion exchange chromatography, adsorption chromatography on cellulose or silica resins, gel-filtration, as well as DNAse, protease or ssRNA-specific RNase treatments. In an alternative embodiment, dsRNA can be purified from isolated virus particles, which can also reduce the amount of impurities.

The maximum size of the target nucleic acid depends on the RNA genome used in the method. For φ6 the theoretical maximum size is 15 kb. The length of the target nucleic acid can be from about 20 bases, preferably from about 50 bases to 15 kilobases, more preferably it is from 50 bases to 5 kilobases, still more preferably from 300 bases to 3 kilobases. The amount of the produced dsRNA is 1 to 5 mg per liter of the culture medium, but may be increased upon optimization.

A Living Cell System for Mass Production of dsRNA

One further object of this invention is a living cell system for mass production of dsRNA.

The system comprises:
a target nucleic acid sequence operably linked with determinants essential for replication by an RNA synthesis apparatus of an RNA virus or another RNA replicon;
a living cell capable of supporting the replication of the RNA virus or other RNA replicon; and
a recovery procedure for recovery of the dsRNA products in a sufficiently pure form.
The cells are preferably either carrier-state or can be transformed into carrier state. The vector is preferably a suicide vector.
“Sufficiently pure” means here that the dsRNA product is as pure as requested for a certain application. The purity may be sufficient for a certain application after the extraction step, when the purity is 80 or 90%. In a certain application several purification steps may be needed until the dsRNA is practically homogenous.
   Kits for Mass Production of dsRNA

One still further object of this invention is a kit for mass production of dsRNA. The kit comprises one or more, preferably at least two of the following items:
a) a vector for transient expression of target nucleic acid in preselected cells that either are carrier-state or can be transformed into carrier state and/or
b) a genetically modified virus into where the target nucleic acid can be introduced; and/or
c) cells that either are carrier-state or can be transformed into carrier state.

The vector is preferably a suicide vector.

Application of the dsRNA Products of this Invention

As an example of the applications of this invention a method is provided for inducing sequence-specific gene silencing effects, such as RNAi, wherein RNA replicons are utilized as vehicles for mass production of heterologous sequences in the dsRNA form in vivo.

This method comprises the steps of:
a) providing nucleic acid target in a form replicable by an RNA-dependent RNA polymerase in a living cell;
b) contacting said replicable form of the nucleic acid target with said polymerase under conditions sufficient for template-directed RNA synthesis, one of the reaction products being necessarily double-stranded (ds) RNA;
c) recovering said dsRNA products in a sufficiently pure form and optionally modifying said products for optimal performance;
d) using said pure, optionally modified dsRNA products to induce sequence-specific gene-silencing effects in eukaryotic systems, such as organisms, cells or cell-free extracts.

The present invention provides a novel strategy for generating double-stranded (ds) RNA triggers suitable for inducing sequence specific gene silencing effects in eukaryotes. A comprehensive description of the sequence specific gene silencing, also referred to as RNA silencing, can be found elsewhere (Baulcombe, 2002; Cogoni, 2001; Hannon, 2002; Vance and Vaucheret, 2001). Briefly, RNA silencing is a group of phenomena in which dsRNA triggers induce sequence-specific downregulation of the expression of target genes in eukaryotic organisms. The form of RNA silencing where dsRNA trigger is introduced into the cell artificially is called RNA interference (RNAi). Several important applications of RNAi have been reported ranging from functional genomics to curing disease (Barstead, 2001; Jacque et al., 2002; Kamath et al., 2003; Lum et al., 2003; McCaffrey et al., 2003; Novina et al., 2002; Pekarik et al., 2003).

In some applications, dsRNA triggers, provided in an isolated form, are administered into living cell or cell-free extracts to induce genesilencing effects. Accordingly, several in vitro methods for producing dsRNA of desired sequence have been reported in the prior art. A large group of such methods comprise the steps of providing two self-complementary single-stranded (ss) RNA and annealing these ssRNAs into a duplex. Alternatively, isolated RNA-dependent RNA polymerases were used to generate dsRNAs from ssRNA templates in vitro (PCT/FI00/01135; WO 01/46396).

In specific embodiments intended for inducing sequence specific gene silencing in inverterbrate animals, fungi, protozoa and plants, extensive dsRNA triggers purified as described above can be used as such. However, in vertebrate animals, long dsRNA may induce a number of unspecific effects, whereas 19-22 nt long dsRNA fragments induce sequence-specific silencing only (McManus and Sharp, 2002). It may therefore be advantageous for embodiments, which involve inducing RNAi in vertebrates or vertebrate cell lines, to fragment long dsRNAs into 19-22 nt pieces. Several fragmentation methods have been described elsewhere including the hydrolysis by ribonucleases DICER and RNase III (Myers et al., 2003; Yang et al., 2002).

The following Examples provide further illustrations of various aspects and embodiments of the present invention. A skilled artisan will appreciate that specific details can be modified without departing from the scope of the invention.

### EXAMPLES

### Example 1

### Introducing Heterologous Sequences into the Genome of dsRNA Virus φ6 and Creating Carrier-State Host Bacteria

### 1.1. Bacterial Strains and Plasmids

Escherichia coli DH5α was used as a host for plasmid propagation and gene engineering. Plasmid pEM35 was produced by inserting the neomycin phosphotransferase (npt) cassette from pUC4K (Pharmacia) at the PstI site of pLM656 (Olkkonen et al., 1990). The correct plasmid encoding the φ6 M segment with the inserted npt gene in the sense orientation was selected using restriction analysis. To construct pEM37, the TfiI-XbaI fragment, containing the φ6 M segment, was excised from pLM656, the ends were filled in using the Klenow fragment of DNA polymerase I, and the blunt fragment was inserted into the pSU 18 vector (chloramphenicol resistance marker; (Bartolome et al., 1991)) at HindIII-Abal sites. To produce pEM38, the β-lactamase (bla) gene was amplified from pUC18 using the primers 5′-TTCACTGCAGATGCATAAGGAAGCATATGAGTATTCAACATTTCCGT-3′ (SEQ ID NO: 1) and 5′-CAAACTGCAGAAGCTTACCAATGCTTAATCAGTGAGGCA-3′ (SEQ ID NO:2) and Pfu DNA polymerase (Stratagene). The resulting PCR fragment was inserted at the PstI site of pEM37 in the sense orientation.

### 1.2. Constructing φ6-npt Carrier-State Cells

The infection of Pseudomonas syringae HB10Y with the wild-type φ6 culminates in cell lysis and release of viral progeny (Mindich, 1988). However, when the kanamycin resistance marker npt was inserted into φ6 M segment, it was possible to select carrier state bacteria on Km-containing medium (Onodera et al., 1992).

We repeated this experiment to obtain a Km-resistant strain HB10Y(φ6-npt). Briefly, purified recombinant φ6 procapsids (PCs) were packaged in vitro with recombinant m⁺ (single-stranded sense copy of φ6 M segment) containing the npt gene (T7 transcript from pEM35 treated with XbaI and mung bean nuclease) and the wild-type l⁺ and s⁺ (single-stranded sense copies of L and S). The packaged ssRNAs were converted into dsRNAs using PC replication in vitro and the particles were coated with φ6 P8 protein to produce infectious nucleocapsids (Bamford et al., 1995). These were used to produce recombinant virus plaques on a P. syringae HB 10Y lawn. Material from one of the plaques (clone #26) was streaked onto LB agar plates containing 30 μg/ml kanamycin (Km) to select carrier-state bacteria HB10Y(φ6-npt) bearing the recombinant virus. These could be stably propagated on Km-containing LB agar or in LB medium without loosing the npt gene, as judged by agarose gel electrophoresis of viral dsRNA and RT-PCR with npt-specific primers 5′-CAAGGAATTCCATGGGCCATATTCAACGGGAAA-3′ (SEQ ID NO:3) and 5′-CCAGGATCCTTTAAAAAAACTCATCGAGCATCAAATGAAACT-3′ (SEQ ID NO:4).

As expected, dsRNA segment M of the φ6-npt virus (M-npt), was longer than wild-type M, whereas φ6-npt L and S segments had regular lengths (FIG. 2A, lanes φ6 and K).

### 1.3. Constructing φ6-bla Carrier-State Cells

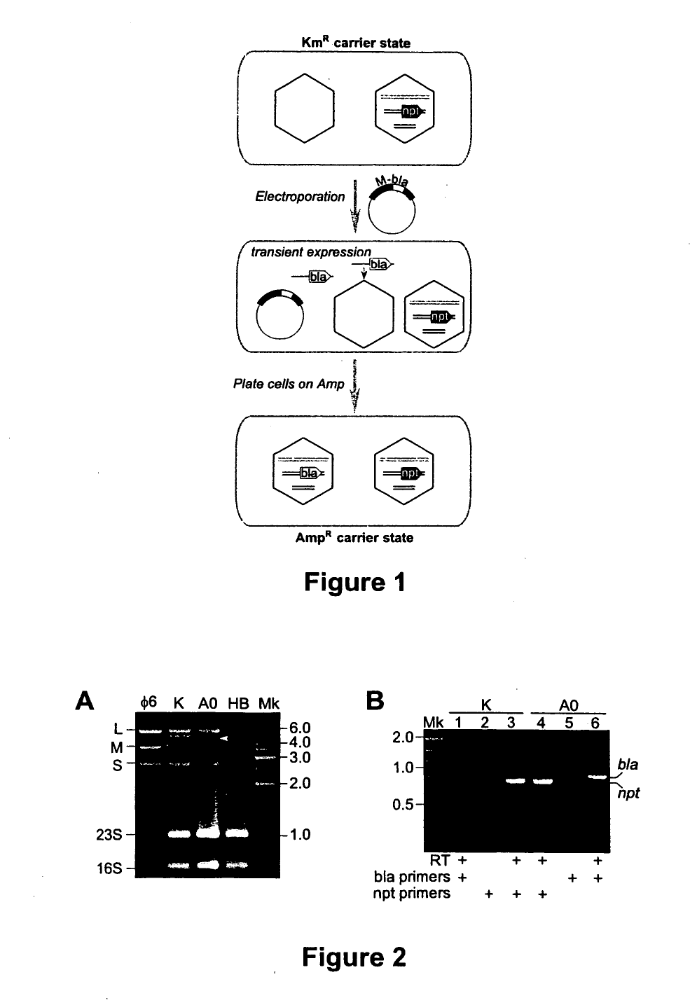

Constructing φ6-npt involved manipulations with purified RNAs and viral procapsids (PCs) in vitro, followed by spheroplast infection (Bamford et al., 1995). To avoid these technical difficulties when preparing φ6-bla virus, we used a plasmid-based strategy (FIG. 1) first developed by Mindich and colleagues (Mindich, 1999b). HB 10Y(+6-npt) cells were transformed with plasmid pEM38 that encodes the φ6 M segment containing the ampicillin resistance marker bla.

For the transformation, electrocompetent HB10Y(φ6-npt) cells were prepared as described (Lyra et al., 1991). These (40 μl) were electroporated with 0.1 mg/ml pEM38. The cell suspension was diluted with 1 ml of LB containing 1 mM MgSO₄, incubated at 28° C. for 2 h, and plated onto LB agar containing 150 μg/ml ampicillin.

pEM38 can not replicate in P. syringae but it can direct transient expression of the recombinant M segment, as previously shown for other E. coli plasmids (Mindich, 1999b). Some of the RNA transcripts can be packaged by PCs, present in the HB 10Y(φ6-npt) cytoplasm, giving rise to φ6-bla virus. Indeed, Amp-resistant colonies (10¹ to 10² μg⁻¹ DNA) appeared after 48-72 h of incubation at 28° C. on pEM38- but not on mock-transformed plates. One of the Amp-resistant clones, which could be stably propagated in the presence of Amp, was used for subsequent experiments. Electrophoretic analysis of the φ6-bla dsRNA genomic segments revealed the presence of two M segment species, M-npt and a new segment, M-bla, migrating between M-npt and wt M (FIG. 2A, lane A0).

### 1.4. Carrier State Bacteria Contain RNA-Encoded Antibiotic Resistance Genes

We carried out RT-PCR analysis to ensure that the bla gene was indeed encoded by φ6-bla rather than by host DNA. The bla PCR product was readily detectable when nucleic acid extracted from HB10Y(φ6-bla) was reverse-transcribed and amplified using bla-specific primers (FIG. 2B, lane 6). However, no product appeared in the control when the RT step was performed without reverse transcriptase (lane 5). This strongly suggests the RNA nature of the bla gene. Using npt-specific primers, we also observed that HB10Y(φ6-bla) bacteria retain detectable amounts of the npt gene (lane 4), consistent with the electrophoretic analysis of HB 10Y(φ6-bla) RNA. As expected, HB10Y(φ6-npt) cells contained only an RNA-encoded npt gene (lanes 1-3).

### Example 2

### Mass Production of dsRNA

2.1. Preparation of Total RNA from Carrier-State Bacteria

Bacterial cells pooled from 20-40 carrier-state colonies or pelleted from 1.5-ml liquid cultures were resuspended in 300 μl of 50 mM Tris-HCl, pH 8.0, 100 mM EDTA, 8% (v/w) sucrose. Lysozyme was added to 1 mg/ml and the mixture was incubated for 5 min at room temperature. Cells were lysed by 1% SDS for 3-5 min. SDS and most of the chromosomal DNA were precipitated by 1.5 M potassium acetate, pH 7.5 on ice. RNA was precipitated from the supernatant fraction by the addition of 0.7 volumes of isopropanol. The RNA pellet was dissolved in 400 μl TE (10 mM Tris-HCl, pH 8.0; 1 mM EDTA), extracted successively with equal volumes of phenol-chloroform and chloroform, and re-precipitated with ethanol. The pellet was washed with 70% ethanol and dissolved in 100 μl of sterile water.

### 2.2. RT-PCR and Cloning of the bla Gene

To obtain cDNA copies of the virus-encoded bla gene, total RNA (1 to 5 μg) from carrier-state bacteria was mixed with 10 μmol of the reverse transcription primer (5′-CTATCGAGCACAGCGCCAACT-3′) (SEQ ID NO:5), denatured by boiling for 1 min and chilled on ice. Reverse transcription was performed using AMV-RT (Sigma) at 45° C. for 1 h as recommended. The bla cDNA was PCR amplified using a mixture of Pfu and Taq DNA polymerases and the primers 5′-CCGAATTCATAAGGAAGCATATGAGTATTCA-3′ (SEQ ID NO:6) and 5′-CAACTTTTACGCTGGTGCTATACAACGACT-3′ (SEQ ID NO:7). HindIII-EcoRI cut PCR products were ligated with a similarly treated pSU 18 vector and transformed into E. coli DH5α. Cloned bla sequences were determined using a commercial automated sequencing facility (MWG-Biotech).

2.3. Gene bla from Ctx-Adapted Carrier State P. syringae Cells Confers Ctx Resistance in E. coli

To characterize the possible effect of cefotaxime selection on the β-lactamase gene, bla cDNA from A0, C1-C4, C7 and C10 passages was cloned into pSU18 (E. coli plasmid containing chloramphenicol (Cm) resistance marker) under control of the lac promoter. E. coli DH5α was transformed with the resulting plasmid libraries and plated onto Cm medium. Because existing cefotaxime-specific β-lactamases are also resistant to ampicillin (Bradford, 2001), we used plates with a low Amp concentration (50 μg/ml) to screen the libraries for clones containing the bla insert. A sufficient amount of β-lactamase was produced from the lac promoter without induction. Plasmids from the Amp-resistant clones (isolated from the master Cm plates) always contained the bla inserts. Conversely, several randomly selected clones that were resistant to Cm but not to Amp were the same size as the pSU18 vector.

We next examined whether E. coli containing pSU 18 with bla inserts originating from φ6-bla are also resistant to Ctx. For this purpose, ˜10⁶ cells were transferred from colonies grown on Cm, -to plates containing 5 or 10 μg/ml Ctx. Of the 50-100 colonies analyzed for each library, 22% of the C1-derived bla clones were indeed resistant to 5 μg/ml Ctx. In the case of C2-, C3-, C4-, C7- and C10-derived libraries, the fraction of Ctx-resistant bla clones was 72, 81, 93, 100 and 100%, respectively, with most of the clones growing in the presence of 5 and 10 μg/ml Ctx. No Ctx-resistant colonies were detected in the A0-derived library.

The obtained constructs in carrier state bacterial cells are propageted with appropriate selection in rich LB gowth medium either in batch cultures, continuous cultures or large scale fermentors. The cells are harvested by centrifugation either using bacth centrifugation or continuous centrifugation. The RNA is extracted and dsRNA separated from cellular RNA as described above.

### REFERENCES

Altamirano, M. M., Blackburn, J. M., Aguayo, C. and Fersht, A. R. (2000) Directed evolution of new catalytic activity using the alpha/beta-barrel scaffold. Nature, 403, 617-22.
Ambler, R. P., Coulson, A. F., Frere, J. M., Ghuysen, J. M., Joris, B., Forsman, M., Levesque, R. C., Tiraby, G. and Waley, S. G. (1991) A standard numbering scheme for the class A beta-lactamases. Biochem J, 276, 269-70.
Amstutz, P., Forrer, P., Zahnd, C. and Pluckthun, A. (2001) In vitro display technologies: novel developments and applications. Curr Opin Biotechnol, 12, 400-5.
Arnold, F. H. (2001) Combinatorial and computational challenges for biocatalyst design. Nature, 409, 253-7.
Bamford, D. H. and Mindich, L. (1980) Electron microscopy of cells infected with nonsense mutants of bacteriophage phi 6. Virology, 107, 222-8.
Bamford, D. H., Ojala, P. M., Frilander, M., Walin, L. and Bamford, J. K. H. (1995) Isolation, purification, and function of assembly intermediates and subviral particles of bacteriophages PRDI and phi6. In Adolph, K. W. (ed.) Methods in molecular genetics. Academic Press, San Diego, Vol. 6, pp. 455-74.
Barstead, R. (2001) Genome-wide RNAi. Curr Opin Chem Biol, 5, 63-6.
Bartolome, B., Jubete, Y., Martinez, E. and de la Cruz, F. (1991) Construction and properties of a family of pACYC184-derived cloning vectors compatible with pBR322 and its derivatives. Gene, 102, 75-8.
Baulcombe, D. (2002) RNA silencing. Curr Biol, 12, R82-4.
Bittker, J. A., Le, B. V. and Liu, D. R. (2002a) Nucleic acid evolution and minimization by nonhomologous random recombination. Nat Biotechnol, 20, 1024-9.
Bittker, J. A., Phillips, K. J. and Liu, D. R. (2002b) Recent advances in the in vitro evolution of nucleic acids. Curr Opin Chem Biol, 6, 367-74.
Bornscheuer, U. T. and Pohl, M. (2001) Improved biocatalysts by directed evolution and rational protein design. Curr Opin Chem Biol, 5, 137-43.
Bradford, P. A. (2001) Extended-spectrum beta-lactamases in the 21st century: characterization, epidemiology, and detection of this important resistance threat. Clin Microbiol Rev, 14, 933-51, table of contents.
Brown, D. and Gold, L. (1995) Selection and characterization of RNAs replicated by Q beta replicase. Biochemistry, 34, 14775-82.
Buck, K. W. (1996) Comparison of the replication of positive-stranded RNA viruses of plants and animals. Adv Virus Res, 47, 159-251.
Burgstaller, P., Jenne, A. and Blind, M. (2002) Aptamers and aptazymes: accelerating small molecule drug discovery. Curr Opin Drug Discov Devel, 5, 690-700.
Carter, P. (2001) Improving the efficacy of antibody-based cancer therapies. Nat Rev Cancer, 1, 118-29.
Chao, L., Rang, C. U. and Wong, L. E. (2002) Distribution of spontaneous mutants and inferences about the replication mode of the RNA bacteriophage phi6. J Virol, 76, 3276-81.
Chen, W. and Georgiou, G. (2002) Cell-Surface display of heterologous proteins: From high-throughput screening to environmental applications. Biotechnol Bioeng, 79, 496-503.
Cobaleda, C. and Sanchez-Garcia, I. (2001) RNase P: from biological function to biotechnological applications. Trends Biotechnol, 19, 406-11.
Coco, W. M., Levinson, W. E., Crist, M. J., Hektor, H. J., Darzins, A., Pienkos, P. T., Squires, C. H. and Monticello, D. J. (2001) DNA shuffling method for generating highly recombined genes and evolved enzymes. Nat Biotechnol, 19, 354-9.
Cogoni, C. (2001) Homology-dependent gene silencing mechanisms in fungi. Annu Rev Microbiol, 55, 381-406.
Cohen, N., Abramov, S., Dror, Y. and Freeman, A. (2001) In vitro enzyme evolution: the screening challenge of isolating the one in a million. Trends Biotechnol, 19, 507-10.
de Feyter, R. and Li, P. (2000) Technology evaluation: HIV ribozyme gene therapy, Gene Shears Pty Ltd. Curr Opin Mol Ther, 2, 332-5.
Domingo, E., Biebricher, C. K., Eigen, M. and Holland, J. J. (2001) Quasispecies and RNA virus evolution: principles and consequences. Landes Bioscience, Georgetown, Tex.
Domingo, E., Escarmis, C., Sevilla, N., Moya, A., Elena, S. F., Quer, J., Novella, I. S, and Holland, J. J. (1996) Basic concepts in RNA virus evolution. Faseb J, 10, 859-64.
Domingo, E., Menendez-Arias, L., Quinones-Mateu, M. E., Holguin, A., Gutierrez-Rivas, M., Martinez, M. A., Quer, J., Novella, I. S, and Holland, J. J. (1997) Viral quasispecies and the problem of vaccine-escape and drug-resistant mutants. Prog Drug Res, 48, 99-128.
Doudna, J. A. and Cech, T. R. (2002) The chemical repertoire of natural ribozymes. Nature, 418, 222-8.
Drake, J. W. and Holland, J. J. (1999) Mutation rates among RNA viruses. Proc Natl Acad Sci USA, 96, 13910-3.
Eigen, M. (1996) On the nature of virus quasispecies. Trends Microbiol, 4, 216-8.
Farci, P., Shimoda, A., Coiana, A., Diaz, G., Peddis, G., Melpolder, J. C., Strazzera, A., Chien, D. Y., Munoz, S. J., Balestrieri, A., Purcell, R. H. and Alter, H. J. (2000) The outcome of acute hepatitis C predicted by the evolution of the viral quasispecies. Science, 288, 339-44.
Farinas, E. T., Bulter, T. and Arnold, F. H. (2001) Directed enzyme evolution. Curr Opin Biotechnol, 12, 545-51.
Forrer, P., Jung, S, and Pluckthun, A. (1999) Beyond binding: using phage display to select for structure, folding and enzymatic activity in proteins. Curr Opin Struct Biol, 9, 514-20.
Fouchier, R. A., Kuiken, T., Schutten, M., Van Amerongen, G., Van Doomum, G. J., Van Den Hoogen, B. G., Peiris, M., Lim, W., Stohr, K. and Osterhaus, A. D. (2003) Aetiology: Koch's postulates fulfilled for SARS virus. Nature, 423, 240.
Gallie, D. R. and Walbot, V. (1990) RNA pseudoknot domain of tobacco mosaic virus can functionally substitute for a poly(A) tail in plant and animal cells. Genes Dev, 4, 1149-57.
Gibbs, M. J., Koga, R., Moriyama, H., Pfeiffer, P. and Fukuhara, T. (2000) Phylogenetic analysis of some large double-stranded RNA replicons from plants suggests they evolved from a defective single-stranded RNA virus. J Gen Virol, 81, 227-33.
Griffiths, A. D. and Tawfik, D. S. (2003) Directed evolution of an extremely fast phosphotriesterase by in vitro compartmentalization. Embo J, 22, 24-35.
Grosshans, H. and Slack, F. J. (2002) Micro-RNAs: small is plentiful. J Cell Biol, 156, 17-21.
Hannon, G. J. (2002) RNA interference. Nature, 418, 244-51.
Harrigan, P. R. and Alexander, C. S. (1999) Selection of drug-resistant HIV. Trends Microbiol, 7, 120-3.
Hoogstraten, D., Qiao, X., Sun, Y., Hu, A., Onodera, S, and Mindich, L. (2000) Characterization of phi8, a bacteriophage containing three double-stranded RNA genomic segments and distantly related to Phi6. Virology, 272, 218-24.
Hudson, P. J. and Souriau, C. (2003) Engineered antibodies. Nat Med, 9, 129-34.
Jacque, J. M., Triques, K. and Stevenson, M. (2002) Modulation of HIV-1 replication by RNA interference. Nature, 418, 435-8.
Jaschke, A. (2001) Artificial ribozymes and deoxyribozymes. Curr Opin Struct Biol, 11, 321-6.
Jeruzalmi, D. and Steitz, T. A. (1998) Structure of T7 RNA polymerase complexed to the transcriptional inhibitor T7 lysozyme. Embo J, 17, 4101-13.
Kamath, R. S., Fraser, A. G., Dong, Y., Poulin, G., Durbin, R., Gotta, M., Kanapin, A., Le Bot, N., Moreno, S., Sohrmann, M., Welchman, D. P., Zipperlen, P. and Ahringer, J. (2003) Systematic functional analysis of the Caenorhabditis elegans genome using RNAi. Nature, 421, 231-7.
Kolkman, J. A. and Stemmer, W. P. (2001) Directed evolution of proteins by exon shuffling. Nat Biotechnol, 19, 423-8.
Kurtzman, A. L., Govindarajan, S., Vahle, K., Jones, J. T., Heinrichs, V. and Patten, P. A. (2001) Advances in directed protein evolution by recursive genetic recombination: applications to therapeutic proteins. Curr Opin Biotechnol, 12, 361-70.
Lai, M. M. (1995) The molecular biology of hepatitis delta virus. Annu Rev Biochem, 64, 259-86.
Leathers, V., Tanguay, R., Kobayashi, M. and Gallie, D. R. (1993) A phylogenetically conserved sequence within viral 3′ untranslated RNA pseudoknots regulates translation. Mol Cell Biol, 13, 5331-47.
Lemon, B. and Tjian, R. (2000) Orchestrated response: a symphony of transcription factors for gene control. Genes Dev, 14, 2551-69.
Lindbo, J. A., Fitzmaurice, W. P. and della-Cioppa, G. (2001) Virus-mediated reprogramming of gene expression in plants. Curr Opin Plant Biol, 4, 181-5.
Long-McGie, J., Liu, A. D. and Schellenberger, V. (2000) Rapid in vivo evolution of a beta-lactamase using phagemids. Biotechnol Bioeng, 68, 121-5.
Lum, L., Yao, S., Mozer, B., Rovescalli, A., Von Kessler, D., Nirenberg, M. and Beachy, P. A. (2003) Identification of Hedgehog pathway components by RNAi in Drosophila cultured cells. Science, 299, 2039-45.
Lyra, C., Savilahti, H. and Bamford, D. H. (1991) High-frequency transfer of linear DNA containing 5′-covalently linked terminal proteins: electroporation of bacteriophage PRD1 genome into Escherichia coli. Mol Gen Genet, 228, 65-9.
Marra, M. A., Jones, S. J., Astell, C. R., Holt, R. A., Brooks-Wilson, A., Butterfield, Y. S., Khattra, J., Asano, J. K., Barber, S. A., Chan, S. Y., Cloutier, A., Coughlin, S. M., Freeman, D., Girn, N., Griffith, O. L., Leach, S. R., Mayo, M., McDonald, H., Montgomery, S. B., Pandoh, P. K., Petrescu, A. S., Robertson, A. G., Schein, J. E., Siddiqui, A., Smailus, D. E., Stott, J. M., Yang, G. S., Plummer, F., Andonov, A., Artsob, H., Bastien, N., Bernard, K., Booth, T. F., Bowness, D., Drebot, M., Fernando, L., Flick, R., Garbutt, M., Gray, M., Grolla, A., Jones, S., Feldmann, H., Meyers, A., Kabani, A., Li, Y., Normand, S., Stroher, U., Tipples, G. A., Tyler, S., Vogrig, R., Ward, D., Watson, B., Brunham, R. C., Krajden, M., Petric, M., Skowronski, D. M., Upton, C. and Roper, R. L. (2003) The Genome Sequence of the SARS-Associated Coronavirus. Science, 1, 1.
McCaffrey, A. P., Nakai, H., Pandey, K., Huang, Z., Salazar, F. H., Xu, H., Wieland, S. F., Marion, P. L. and Kay, M. A. (2003) Inhibition of hepatitis B virus in mice by RNA interference. Nat Biotechnol, 12, 12.
McManus, M. T. and Sharp, P. A. (2002) Gene silencing in mammals by small interfering RNAs. Nat Rev Genet, 3, 737-47.
Miller, W. A. and Koev, G. (1998) Getting a handle on RNA virus recombination. Trends Microbiol, 6, 421-3.
Mindich, L. (1988) Bacteriophage phi 6: a unique virus having a lipid-containing membrane and a genome composed of three dsRNA segments. Adv Virus Res, 35, 137-76.
Mindich, L. (1999a) Precise packaging of the three genomic segments of the double-stranded-RNA bacteriophage phi6. Microbiol Mol Biol Rev, 63, 149-60.
Mindich, L. (1999b) Reverse genetics of dsRNA bacteriophage phi 6. Adv Virus Res, 53, 341-53.
Mindich, L., MacKenzie, G., Strassman, J., McGraw, T., Metzger, S., Romantschuk, M. and Bamford, D. (1985) cDNA cloning of portions of the bacteriophage phi 6 genome. J. Bacteriol, 162, 992-9.
Mindich, L., Qiao, X., Qiao, J., Onodera, S., Romantschuk, M. and Hoogstraten, D. (1999) Isolation of additional bacteriophages with genomes of segmented double-stranded RNA. J. Bacteriol, 181, 4505-8.
Myers, J. W., Jones, J. T., Meyer, T. and Ferrell, J. E. (2003) Recombinant Dicer efficiently converts large dsRNAs into siRNAs suitable for gene silencing. Nat Biotechnol, 21, 324-8.
Negroni, M. and Buc, H. (2001) Mechanisms of retroviral recombination. Annu Rev Genet, 35, 275-302.
Nichol, S. T., Arikawa, J. and Kawaoka, Y. (2000) Emerging viral diseases. Proc Natl Acad Sci USA, 97, 12411-2.
Nixon, A. E., Firestine, S. M., Salinas, F. G. and Benkovic, S. J. (1999) Rational design of a scytalone dehydratase-like enzyme using a structurally homologous protein scaffold. Proc Natl Acad Sci USA, 96, 3568-71.
Novina, C. D., Murray, M. F., Dykxhoorn, D. M., Beresford, P. J., Riess, J., Lee, S. K., Collman, R. G., Lieberman, J., Shankar, P. and Sharp, P. A. (2002) siRNA-directed inhibition of HIV-1 infection. Nat Med, 8, 681-6.
Olkkonen, V. M. and Bamford, D. H. (1989) Quantitation of the adsorption and penetration stages of bacteriophage phi 6 infection. Virology, 171, 229-38.
Olkkonen, V. M., Gottlieb, P., Strassman, J., Qiao, X. Y., Bamford, D. H. and Mindich, L. (1990) In vitro assembly of infectious nucleocapsids of bacteriophage phi 6: formation of a recombinant double-stranded RNA virus. Proc Natl Acad Sci USA, 87, 9173-7.
Olsthoom, R. C., Mertens, S., Brederode, F. T. and Bol, J. F. (1999) A conformational switch at the 3′ end of a plant virus RNA regulates viral replication. Embo J, 18, 4856-64.
O'Neil, K. T. and Hoess, R. H. (1995) Phage display: protein engineering by directed evolution. Curr Opin Struct Biol, 5, 443-9.
Onodera, S., Olkkonen, V. M., Gottlieb, P., Strassman, J., Qiao, X. Y., Bamford, D. H. and Mindich, L. (1992) Construction of a transducing virus from double-stranded RNA bacteriophage phi6: establishment of carrier states in host cells. J. Virol, 66, 190-6.
Onodera, S., Qiao, X., Gottlieb, P., Strassman, J., Frilander, M. and Mindich, L. (1993) RNA structure and heterologous recombination in the double-stranded RNA bacteriophage phi 6. J. Virol, 67, 4914-22.
Onodera, S., Qiao, X., Qiao, J. and Mindich, L. (1995) Acquisition of a fourth genomic segment in bacteriophage phi 6, a bacteriophage with a genome of three segments of dsRNA. Virology, 212, 204-12.
Onodera, S., Qiao, X., Qiao, J. and Mindich, L. (1998) Directed changes in the number of double-stranded RNA genomic segments in bacteriophage phi6. Proc Natl Acad Sci USA, 95, 3920-4.
Onodera, S., Sun, Y. and Mindich, L. (2001) Reverse genetics and recombination in Phi8, a dsRNA bacteriophage. Virology, 286, 113-8.
Orencia, M. C., Yoon, J. S., Ness, J. E., Stemmer, W. P. and Stevens, R. C. (2001) Predicting the emergence of antibiotic resistance by directed evolution and structural analysis. Nat Struct Biol, 8, 238-42.
Palese, P. (1998) RNA virus vectors: where are we and where do we need to go? Proc Natl Acad Sci USA, 95, 12750-2.
Pasloske, B. L. (2001) Ribonuclease inhibitors. Methods Mol Biol, 160, 105-11.
Pekarik, V., Bourikas, D., Miglino, N., Joset, P., Preiswerk, S, and Stoeckli, E. T. (2003) Screening for gene function in chicken embryo using RNAi and electroporation. Nat Biotechnol, 21, 93-6.
Petrounia, I. P. and Arnold, F. H. (2000) Designed evolution of enzymatic properties. Curr Opin Biotechnol, 11, 325-30.
Pohorille, A. and Deamer, D. (2002) Artificial cells: prospects for biotechnology. Trends Biotechnol, 20, 123-8.
Poranen, M. M., Paatero, A. O., Tuma, R. and Bamford, D. H. (2001) Self-assembly of a viral molecular machine from purified protein and RNA constituents. Mol Cell, 7, 845-54.
Qiao, X., Qiao, J. and Mindich, L. (1997) An in vitro system for the investigation of heterologous RNA recombination. Virology, 227, 103-10.
Qiao, X., Qiao, J., Onodera, S. and Mindich, L. (2000) Characterization of phi 13, a bacteriophage related to phi 6 and containing three dsRNA genomic segments. Virology, 275, 218-24.
Quemeneur, E., Moutiez, M., Charbonnier, J. B. and Menez, A. (1998) Engineering cyclophilin into a proline-specific endopeptidase. Nature, 391, 301-4.
Rader, C. and Barbas, C. F., 3rd. (1997) Phage display of combinatorial antibody libraries. Curr Opin Biotechnol, 8, 503-8.
Robbins, P. D., Tahara, H. and Ghivizzani, S. C. (1998) Viral vectors for gene therapy. Trends Biotechnol, 16, 35-40.
Robertson, M. P. and Ellington, A. D. (2001) In vitro selection of nucleoprotein enzymes. Nat Biotechnol, 19, 650-5.
Sachs, A. B. (2000) Cell cycle-dependent translation initiation: IRES elements prevail. Cell, 101, 243-5.
Sachs, A. B. and Buratowski, S. (1997) Common themes in translational and transcriptional regulation. Trends Biochem Sci, 22, 189-92.
Schatz, P. J., Cull, M. G., Martin, E. L. and Gates, C. M. (1996) Screening of peptide libraries linked to lac repressor. Methods Enzymol, 267, 171-91.
Selifonova, O., Valle, F. and Schellenberger, V. (2001) Rapid evolution of novel traits in microorganisms. Appl Environ Microbiol, 67, 3645-9.
Soumillion, P. and Fastrez, J. U.-h.w.s.c.s.a.B. V.-K. C.-D.b.f.e.e.f.b.c.c.a.f. (2001) Novel concepts for selection of catalytic activity. Current Opinion in Biotechnology, 12, 387-394.
Stemmer, W. P. (1994a) DNA shuffling by random fragmentation and reassembly: in vitro recombination for molecular evolution. Proc Natl Acad Sci USA, 91, 10747-51.
Stemmer, W. P. (1994b) Rapid evolution of a protein in vitro by DNA shuffling. Nature, 370, 389-91.
Tawfik, D. S. and Griffiths, A. D. (1998) Man-made cell-like compartments for molecular evolution. Nat Biotechnol, 16, 652-6.
Theil, E. C. (1993) The IRE (iron regulatory element) family: structures which regulate mRNA translation or stability. Biofactors, 4, 87-93.
Trower, M. K. (1996) In vtro mutagenesis protocols. Humana Press, Totowa, N. J. Vagner, S., Galy, B. and Pyronnet, S. (2001) Irresistible IRES. Attracting the translation machinery to internal ribosome entry sites. EMBO Rep, 2, 893-8.
van Roessel, P. and Brand, A. H. (2002) Imaging into the future: visualizing gene expression and protein interactions with fluorescent proteins. Nat Cell Biol, 4, E15-20.
Vance, V. and Vaucheret, H. (2001) RNA silencing in plants—defense and counterdefense. Science, 292, 2277-80.
White, L. K., Wright, W. E. and Shay, J. W. (2001) Telomerase inhibitors. Trends Biotechnol, 19, 114-20.
Wickner, R. B. (1996) Prions and RNA viruses of Saccharomyces cerevisiae. Annu Rev Genet, 30, 109-39.
Wilson, D. S. and Szostak, J. W. (1999) In vitro selection of functional nucleic acids. Annu Rev Biochem, 68, 611-47.
Winter, G. and Harris, W. J. (1993) Humanized antibodies. Immunol Today, 14, 243-6.
Wittrup, K. D. U.-h.w.s.c.s.a.B. V.-K. C.-F.c.c.f.c.a. (2001) Protein engineering by cell-surface display. Current Opinion in Biotechnology, 12, 395-399.
Yang, D., Buchholz, F., Huang, Z., Goga, A., Chen, C. Y., Brodsky, F. M. and Bishop, J. M. (2002) Short RNA duplexes produced by hydrolysis with Escherichia coli RNase III mediate effective RNA interference in mammalian cells. Proc Natl Acad Sci US A, 99, 9942-7.
Zeenko, V. V., Ryabova, L. A., Spirin, A. S., Rothnie, H. M., Hess, D., Browning, K. S. and Hohn, T. (2002) Eukaryotic elongation factor 1A interacts with the upstream pseudoknot domain in the 3′ untranslated region of tobacco mosaic virus RNA. J Virol, 76, 5678-91.
Zhao, H., Chockalingam, K. and Chen, Z. (2002) Directed evolution of enzymes and pathways for industrial biocatalysis. Curr Opin Biotechnol, 13, 104-10.

1. A method for mass production of dsRNA, which comprises:
a) providing nucleic acid target in a form replicable by an RNA-dependent RNA polymerase;
b) incorporating the nucleic acid target into the genome of an RNA virus or other RNA replicon encoding said polymerase, where said nucleic acid target is replicated by the polymerase encoded by the RNA virus or other RNA replicon under conditions sufficient for template-directed RNA synthesis in a living cell, one of the reaction products being necessarily double-stranded (ds) RNA; and
c) recovering said dsRNA products in a sufficiently pure form.

2. The method according to claim 1, wherein said nucleic acid target encodes a polypeptide or is equivalent to a noncoding region in the genome of a desired organism.

3. The method according to claim 1, wherein the nucleic acid target is operably linked with determinants essential for detectable replication by the polymerase.

4. The method according to claim 1, wherein the polymerase is a genetically modified or wild-type polymerase.

5. The method according to claim 1, wherein the RNA virus or other RNA replicon is genetically modified or wild-type.

6. The method according to claim 1, wherein the RNA replicon is RNA virus-like particle, viroid or RNA-based autonomous genetic element.

7. The method according to claim 1, wherein the nucleic acid encoding the polymerase and the nucleic acid target are distinct nucleic acids.

8. The method according to claim 1, wherein the RNA virus is an RNA bacteriophage.

9. The method according to claim 7, wherein the RNA virus is from a member of the Cystovilidae family, preferably from a bacteriophage selected from the group comprising φ6, φ7, +8, φ9, φ10, φ11, φ12, φ13 and φ14, most preferably from bacteriophage φ6.

10. The method according to claim 1, wherein the replicable form of the nucleic acid target is replicated in a prokaryotic cell, preferably in a gram-negative bacterial cell, more preferably in a bacterial cell selected from the group comprising Pseudomonas sp., Escherichia sp. and Salmonella sp., most preferably in a cell of Pseudomonas syringae.

11. The method according to claim 1, wherein the replicable form of the nucleic acid target is replicated in a eukaryotic cell, such as mammalian, insect, plant or yeast cell.

12. The method according to claim 1, wherein the nucleic acid target is delivered into the living cell using a suicide vector, preferably a DNA vector, most preferably a DNA plasmid.

13. The method according to claim 1, wherein a suicide vector, comprising a target nucleic acid operably linked with sequences sufficient for detectable replication by the viral replication apparatus, is used to incorporate said nucleic acid target into the genome of said RNA virus.

14. A system for mass production of dsRNA, which comprises:
a target nucleic acid sequence operably linked with determinants essential for replication by an RNA synthesis apparatus of an RNA virus or another RNA replicon;
a living cell capable of supporting the replication of the RNA virus or other RNA replicon; and
a recovery procedure for recovery of the dsRNA products in a sufficiently pure form.

15. The system according to claim 14, wherein the living cell is a carrier-state cell or can be transformed into carrier state.

16. The system according to claim 14, wherein the nucleic acid target is provided in a suicide vector.

17. The system according to claim 14, wherein the RNA-dependent RNA polymerase in the RNA synthesis apparatus originates from a dsRNA virus or a dsRNA replicon.

18. The system according to claim 14, wherein the RNA-dependent RNA polymerase in the RNA synthesis apparatus originates from the C)ystoviridae family, preferably from a bacteriophage selected from the group comprising φ6, φ7, φ8, φ9, φ10, φ11, φ12, φ13, φ14, most preferably from bacteriophage φ6.

19. The system according to claim 14, wherein the living cell is a prokaryotic cell, preferably a gram-negative bacterial cell, more preferably the bacterial cell is selected from the group comprising Pseudomonas sp., Escherichia sp. and Salmonella sp., most preferably the bacterium is Pseudomonas syringae.

20. A kit for mass production of dsRNA, wherein the kit comprises:
a) a vector for transient expression of target nucleic acid in preselected cells that either are carrier-state or can be transformed into carrier state and/or
b) a genetically modified virus into where the target nucleic acid can be introduced; and/or
c) cells that either are carrier-state or can be transformed into carrier state.

21. A method for inducing sequence-specific gene silencing effects in eukaryotic systems, the method comprising:
a) providing nucleic acid target in a form replicable by an RNA-dependent RNA polymerase;
b) incorporating the nucleic acid target into the genome of an RNA virus or other RNA replicon, where said nucleic acid is replicated by the polymerase encoded by the RNA virus or other RNA replicon under conditions sufficient for template-directed RNA synthesis in a living cell, one of the reaction products being necessarily double-stranded (ds) RNA;
c) recovering said dsRNA products in a sufficiently pure form and optionally modifying said products for optimal performance;
d) using said pure, optionally modified, dsRNA products to induce sequence-specific gene-silencing effects in eukaryotic systems, such as organisms, cells or cell-free extracts.

22. The method according to claim 21, wherein the RNA-dependent RNA polymerase originates from a dsRNA virus or a dsRNA replicon.

23. The method according to claim 21, wherein the dsRNA virus is from the Cystoviridae family, preferably from a bacteriophage selected from the group comprising φ6, φ7, φ8, φ9, φ10, φ11, φ12, φ13, φ14, most preferably from bacteriophage φ6.

24. The method according to claim 21, wherein the living cell is a prokaryotic cell, preferably a gram-negative bacterial cell, more preferably the bacterial cell is selected from the group comprising Pseudomonas sp., Escherichia sp. and Salmonella sp., most preferably the bacterium is Pseudomonas syringae.

25. The method according to claim 21, wherein the optional step of modifying for optional performance is fragmenting dsRNA with dsRNA-specific ribonucleases, preferably RNase III, Dicer, or derivatives thereof.

26. The method according to claim 21, wherein the target nucleic acid is provided in a suicide vector.

27. The method according to claim 21, wherein the dsRNA products are used to induce sequence-specific gene-silencing effects in invertebrate animal systems, preferably of insect or nematode origin, most preferably from Drosophila melanogaster or Caenorhabdits elegans origin.

28. The method according to claim 21, wherein the dsRNA products are used to induce sequence-specific gene-silencing effects in vertebrate animal systems, preferably of mammalian origin, most preferably of human or mouse origin.


SEQUENCE LISTING (7 sequences)

SEQ ID NO: 1 - DNA, 47 bp, Artificial Sequence
  primer
ttcactgcagatgcataaggaagcatatgagtattcaacatttccgt

SEQ ID NO: 2 - DNA, 39 bp, Artificial Sequence
  primer
caaactgcagaagcttaccaatgcttaatcagtgaggca

SEQ ID NO: 3 - DNA, 33 bp, Artificial Sequence
  primer
caaggaattccatgggccatattcaacgggaaa

SEQ ID NO: 4 - DNA, 42 bp, Artificial Sequence
  primer
ccaggatcctttaaaaaaactcatcgagcatcaaatgaaact

SEQ ID NO: 5 - DNA, 21 bp, Artificial Sequence
  primer
ctatcgagcacagcgccaact

SEQ ID NO: 6 - DNA, 31 bp, Artificial Sequence
  primer
ccgaattcataaggaagcatatgagtattca

SEQ ID NO: 7 - DNA, 30 bp, Artificial Sequence
  primer
caacttttacgctggtgctatacaacgact